(19) United States
(12) Patent Application Publication
(10) Pub. No.: US 2008/0306312 A1
(43) Pub. Date: Dec. 11, 2008

(54) Process for Obtaining Alkoxides

(75) Inventors: Bernd Eck, Viernheim (DE); Matthias Rauls, Ludwigshafen (DE); Hermann Josef Feise, Kleinniedesheim (DE); Thomas Letzelter, Annweiler (DE); Josef Guth, Freinsheim (DE)

Correspondence Address: CONNOLLY BOVE LODGE & HUTZ LLP, 1875 EYE STREET, N.W., SUITE 1100, WASHINGTON, DC, 20006, US

(73) Assignee: BASF SE, Ludwigshafen (DE)

(21) Appl. No.: 12/158,544

(22) PCT Filed: Dec. 13, 2006
(86) PCT No.: PCT/EP06/69630

§ 371 Date: Jun. 20, 2008

(30) Foreign Application Priority Data
Dec. 13, 2005 (DE) 10-2005-062-654.8

(51) Int. Cl. C07C 29/78

(52) U.S. Cl. 568/851; 568/700

(57) ABSTRACT

Alkoxides are isolated by crystallization of an alcohol from a solution in the corresponding alcohol, with the alkoxide being crystallized out at a pressure of at least 3 bar abs. and a temperature of at least 120° C.

The present invention relates to a method of isolating alkoxides by crystallization.

Alkoxides are derived from alcohols R—OH by replacement of the hydrogen atom of the hydroxy group by a metal M. They are thus formally metal salts R—OM of the alcohols R—OH. Alkoxides, their preparation and use are well known. Most widespread are the alkali metal alkoxides. Alkali metal alkoxides (systematic name: “Alkali metal alkanolates”) are well-known reagents in organic chemistry. They are used where strong bases are required as reactants and as catalysts for particular reactions. Virtually only aliphatic alkali metal alkoxides of lithium, sodium and potassium having from 1 to 4 carbon atoms in the alkyl radical of the alcohol, in particular lithium, sodium and potassium methoxide, ethoxide, n-propoxide, isopropoxide, n-butoxide and tert-butoxide, are prepared and used in relatively large amounts. Methods of preparing alkali metal alkoxides have been known for a long time. A general overview of aliphatic alkali metal alkoxides, their preparation and use is given, for example, in Ullmann's Encyclopedia of Industrial Chemistry, Sixth Edition, Volume 2, WILEY-VCH Verlag GmbH & Co. KGaA, Weinheim 2003 (ISBN 3-527-30385-5), as item 4. under the keyword “Alcohols, Aliphatic”. Known methods of preparing alkali metal alkoxides are the direct reaction of alkali metal with alcohol and the reaction of alkali metal hydroxide with alcohol combined with removal of the resulting water by distillation, as described, for example, in the German Patent DE 519 443 or in another embodiment in WO 01/42 178 A1. The reaction of alkali metal hydroxide with alcohol combined with removal of the by-product water is comparatively energy-intensive since the by-product water can only be separated off as a small proportion in a large amount of alcohol which is distilled off and needs to be recirculated to the reaction. In some such process variants, a solvent or entrainer circuit is also made necessary by the distillation, as, for instance, in the processes described in the German Patent No. 505 474, in U.S. Pat. No. 1,816,843 or in the French Patent No. 1 089 101. A particularly complicated specific process for preparing alkoxides of higher alcohols which cannot be obtained by direct reaction is the reaction of alkali metal methoxide or alkali metal ethoxide with the higher alcohol combined with removal of methanol or ethanol by distillation.

The direct reaction of an alkali metal with an alcohol is a simple and economical and therefore industrially most widespread way of preparing alkali metal alkoxides. The reactivity of the alkali metals increases in the order lithium, sodium, potassium, rubidium and cesium, and the reactivity of the alcohols decreases with the molecular weight of the alcohol and the degree of branching of the alkyl radical. The reaction is often carried out using a dispersion of the alkali metal in an inert solvent or particularly conveniently using alkali metal amalgam. An overview of the industrial preparation of alkali metal alkoxides from alkali metal amalgam and reaction apparatuses used for this purpose is given by, for example: R. B. MacMullin in: “By-Products of Amalgam-type Chlorine Cell”, Chemical Engineering Progress September 1950, pp. 440-455, 448. A number of specific embodiments of these processes for preparing alkali metal alkoxides by reaction of alkali metal or alkali metal amalgam with alcohol are known, For example, these reactions can be accelerated by use of catalysts, as described in WO 99/65 849 A1, EP 1 195 369 A1 or EP 153 3291 A1. Methods of purifying the crude product are likewise known, for example the methods of removing mercury described in WO 2004/048 624 A1 and WO 2004/048 625 A1.

It is common to all processes for preparing alkali metal alkoxides by reaction of alkali metal with alcohol that unreacted or excess alcohol firstly remains in the crude product as “alcohol of crystallization” in an amount of from one to three mol per mol of alkoxide, which is undesirable for many uses of alkoxides in which protic compounds such as alcohols interfere.

Various methods of avoiding or reducing the amount of this alcohol of crystallization are known. For example, some processes are carried out using a substoichiometric amount of alcohol or a stoichiometric amount of the alcohol, with an inert solvent or suspension medium or even without this, which requires the difficult handling of highly reactive unreacted alkali metal residues and is therefore very unsatisfactory. For this reason, it is usual to employ an at least stoichiometric amount of alcohol. The reaction of stoichiometric amounts of alcohol and alkali metal in an inert solvent or suspension medium which is customary in the laboratory is likewise unsatisfactory in industry. Firstly, it requires an additional outlay for the separation and recirculation of the inert solvent, and in addition alkali metal alkoxide tends in this environment to form encrustations on the alkali metal, so that the alcohol easily forms alkoxide comprising alcohol of crystallization with this alkoxide and unreacted alkali metal remains. This process thus requires an extraordinarily high input of mechanical energy to continually disperse alkali metal and alkoxide including the shearing-off of the alkoxide crusts, which becomes all the more difficult and uneconomical the larger the apparatus used. DD 298 502 A5 discloses an alternative process in which the reaction of alkali metal and alcohol is carried out in a boiling inert solvent whose boiling point is above the temperature at which the alcohol of crystallization is given off from the alkoxide. This is likewise unsatisfactory since, firstly, only relatively small amounts of alcohol are present in vapor form in the reactor and the space-time yield is thus reduced and, secondly, the greatest possible outlay is necessary in the condensation and recirculation of alcohol and also solvent.

Alkoxides are therefore usually prepared in the form of a solution in the corresponding alcohol and are subsequently freed of the alcohol. For this purpose, the solvent is evaporated, resulting in alkoxide comprising alcohol of crystallization precipitating from the solution in crystalline form (crystallizing out). The alcohol of crystallization can also be removed by use of a sufficiently high temperature and, if desired, also application of a vacuum (“calcination”). In such a procedure, the solid product has to be continuously kept in motion in order to achieve uniform freedom from alcohol of crystallization and to prevent formation of lumps.

Methods and apparatuses for crystallization, including methods and apparatuses for crystallization by evaporation of a solvent have been generally known for a long time. Ullmann's Encyclopedia of Industrial Chemistry, Sixth Edition, Wiley-VCH, Weinheim (ISBN 3527-30385-5), gives a general overview of such methods under the keyword “Crystallization and Precipitation” (Volume 10, published 2003). A. S. Myerson (editor): “Handbook of Industrial Crystallization”, 2nd edition, Butterworth-Heinemann, Boston 2002 (ISBN 0-7506-7012-6) and A. Mersmann (editor): “Crystallization Technology Handbook”, 2nd edition, Dekker, New York 2001 (ISBN 0-8247-0528-9) give an overview of technical knowledge in this field.

The abovementioned WO 99/65 849 A1 and also CN 1 239 714 A1 describe such processes in paddle dryers. U.S. Pat. No. 3,587,704 discloses a complicated process for isolating pulverulent sodium alkoxide from methanol in a thin film evaporator with mechanical distribution of the solution to be evaporated and a downstream cyclone. The product of such processes has the disadvantage that it has a very high fines content (particles having a diameter of less than 10 microns), i.e. the alkali metal alkoxide produces a great deal of dust. This is due, firstly, to the mechanical stress during calcination and, secondly, to the lattice structure of the crystals being disrupted by effects such as channel formation through to rupture of the crystals as a result of the alcohol of crystallization being given off from the solid, crystalline alkoxide. The high fines content increases the cohesiveness of the bulk product. Apart from the fact that emptying of containers is made more difficult in this way, appropriate protective measures against the highly corrosive dust are also necessary.

EP 1 306 125 A2 teaches a method of producing alkali metal and alkaline earth metal alkoxide granules by fluidized-bed spray granulation of alkoxide solutions. Here, fluidization of the product in the fluidized bed by means of appropriately large gas streams is necessary here. In addition, a further step for precipitation of fine dust together with recirculation to the granulation is necessary. Both are economically disadvantageous. Furthermore, it is not at all certain that under the process conditions disclosed (e.g. 70-135° C. at atmospheric pressure for the granulation of sodium methoxide) the methanol of crystallization is removed completely.

There is a continuing need for improved methods, in particular methods of producing, in a simple and economical way, alkoxides which are low in dust and are easy to handle.

We have accordingly found a method of isolating alkoxides by crystallization from a solution in the corresponding alcohol, wherein the alkoxide is crystallized out at a pressure of at least 3 bar abs. and a temperature of at least 120° C.

The method of the invention leads to the formation of a product having a narrow particle size distribution and a very low fines content. The product can be handled readily and does not tend to cake. Compared to known simple methods, no additional apparatuses or process steps are necessary. A particular advantage of the method of the invention is that alkoxide which is free of alcohol of crystallization crystallizes out directly, so that a subsequent calcination step and the resulting mechanical stresses on the crystals can be avoided.

In the method of the invention, the alkoxide is crystallized out at a pressure of generally at least 3 bar abs., preferably at a pressure of at least 3.5 bar abs. and particularly preferably at a pressure of at least 4 bar abs. The pressure to be employed in the method of the invention is in principle not subject to any upper limit. However, for economic reasons, in particular the rapidly increasing capital and operating costs for pressure apparatuses designed for higher pressures, an upper limit to the pressure given by the economic boundary conditions of the individual case is chosen. Furthermore, this upper limit is chosen so that a satisfactory crystallization result is achieved at the pressure set and the temperature set. These values which are dependent on the system-specific thermodynamic boundary conditions (dependence of the solubility of the alkoxide on temperature and pressure) can for a given alcohol/alkoxide system either be taken from a database or be readily determined by means of a few routine tests. For example, the method is implemented at a pressure of not more than 325 bar abs., preferably not more than 100 bar abs. and particularly preferably not more than 15 bar abs., for example not more than 10 bar abs. or not more than 6 bar abs. The pressures indicated above are absolute values (“abs.”), i.e. not “gauge pressure” figures (“barg; barg=bar abs.−1).

In the method of the invention, it is usual to set a temperature of at least 120° C., preferably a temperature of at least 125° C. and particularly preferably at least 130° C. As in the case of the pressure, the upper limit to the temperature range to be set is determined by economic boundary conditions and by the solubility of the alkoxide in the alcohol. In general, a temperature of not more than 250° C., preferably not more than 200° C. and particularly preferably not more than 180° C., is set.

The method of the invention is used to isolate alcohols from a solution in the corresponding alcohol. In particular, the process is suitable for the isolation of alkoxides of aliphatic alcohols, especially those having a straight-chain or branched hydrocarbon radical having from one to 8 carbon atoms. Preference is given to using primary, secondary or tertiary alcohols having from one to 5 carbon atoms, particularly preferably those having from one to four carbon atoms. Examples of alcohols used in the method of the invention are methanol, ethanol, n-propanol, isopropanol, n-butanol, 2-butanol (sec-butanol), 2-methyl-1-propanol (isobutanol), 2-methyl-2-propanol (tert-butanol), 1-, 2- or 3-pentanol, neopentanol, tert-pentanol, hexanol, heptanol and octanol. Very particular preference is given to methanol. The corresponding alkoxides are isolated.

The metal in the alkoxide which is isolated by means of the method of the invention can be any metal which forms an alkoxide with the alcohol selected. Preference is given to the alkali metals lithium, sodium, potassium, rubidium and cesium and also the alkaline earth metals beryllium, calcium, magnesium, strontium and barium. Particular preference is given to the alkali metals and very particular preference is given to sodium.

The process of the invention is consequently particularly preferably used to isolate lithium, sodium and potassium methoxide, ethoxide, n-propoxide, isopropoxide, n-butoxide, tert-butoxide, 1-, 2- or 3-pentoxide, neopentoxide, tert-pentoxide, hexoxide, heptoxide or octoxide (trivial names in each case “-ylate” instead of “-oxide”), very particularly preferably sodium methoxide.

To carry out the method of the invention, a solution of the alkoxide to be isolated in the corresponding alcohol is firstly produced in a known manner, for example by reacting the metal or metal amalgam with the alcohol. The solution can, if necessary, be freed of impurities in a known manner, for instance in the preparation by reaction of the metal amalgam with alcohol be freed of mercury by the known distillation or filtration methods, or by decantation or filtration of unreacted impurities of the metal, before the alkoxide is crystallized out.

The alkoxide is crystallized out from this solution by evaporation in a customary apparatus. It is possible to use any apparatuses suitable for crystallization by evaporation which is designed for the pressure to be employed according to the invention and for the temperature to be employed according to the invention. Suitable apparatuses for carrying out the method of the invention are, for example, paddle dryers, stirred vessels or forced circulation crystallizers, guide tube crystallizers or Oslo crystallizers. The method of the invention is preferably carried out in a paddle dryer.

It is possible for the alkoxide to be fractionally crystallized from the solution, but this is generally necessary only when the starting materials used for preparing the alkoxide are unusually heavily contaminated.

Crystallization methods and parameters are, unless explicitly indicated here, known like crystallizers, cf., for example, the abovementioned compendia on crystallization, which are hereby expressly incorporated by reference, and the abovementioned prior art on previously known methods of isolating alkoxides, which are likewise expressly incorporated by reference.

### EXAMPLES

### Comparative Example 1

In a paddle dryer having a volume of 300 l, 30% strength by weight sodium methoxide solution in methanol was evaporated under atmospheric pressure by heating to just above the boiling point of methanol over a period of 4 hours. After complete removal of the solvent, the temperature was increased to 150° C. to drive off the methanol of crystallization from the sodium methoxide. The solid obtained in this way had a mean particle size of 95 microns, and 9% by weight of the solid was made up of particles having a diameter of less than 10 microns. The product displayed severe dust formation (dust number of about 110) and tended to cake even in relatively small containers. (Methods of determining the dust number are known, cf., for example, the overview given by F. Hamelmann and E. Schmidt, Chemie Ingenieur Technik 74 (12) (2002) 1666-1676.)

### Example 1

Comparative example 1 was repeated but with a pressure of 4.5 bar abs. and a temperature of a constant 140° C. being set. The solid obtained after complete removal of the methanol was sodium methoxide which was free of methanol of crystallization and had a mean particle size of 185 microns. Only 2% by weight of the particles had a diameter of less than 10 microns. The product displayed considerably less dust formation than the product of Comparative example 1 (dust number 42) and remained free-flowing in containers.

1. A method of isolating alkoxides by crystallization from a solution in the corresponding alcohol, wherein the alkoxide is crystallized out at a pressure of at least 3 bar abs. and a temperature of at least 120° C.

2. The method according to claim 1, wherein a pressure of at least 3.5 bar abs. is employed.

3. The method according to claim 2, wherein a pressure of at least 4 bar abs. is employed.

4. The method according to claim 1, wherein a temperature of at least 125° C. is employed.

5. The method according to claim 4, wherein a temperature of at least 130° C. is employed.

6. The method according to claim 1, wherein a pressure of at least 4 bar abs. and a temperature of at least 130° C. are employed.

7. The method according to claim 1, wherein an alkali metal alkoxide is isolated.

8. The method according to claim 7, wherein a sodium alkoxide is isolated.

9. The method according to claim 8, wherein sodium methoxide is isolated.

10. The method according to claim 6, wherein an alkali metal alkoxide is isolated.

11. The method according to claim 10, wherein sodium methoxide is isolated.
